(19) United States
(12) Patent Application Publication
(10) Pub. No.: US 2013/0190589 A1
(43) Pub. Date: Jul. 25, 2013

(54) MULTIPLE PEAK ANALYSIS IN A PHOTOACOUSTIC SYSTEM

(71) Applicants: Bo Chen, Louisville, CO (US); Youzhi Li, Longmont, CO (US)

(72) Inventors: Bo Chen, Louisville, CO (US); Youzhi Li, Longmont, CO (US)

(73) Assignee: Nellcor Puritan Bennett LLC, Boulder, CO (US)

(21) Appl. No.: 13/357,839

(22) Filed: Jan. 25, 2012

(51) Int. Cl. A61B 5/05

(52) U.S. Cl. 600/407

(57) ABSTRACT

A physiological monitoring system may use photoacoustic sensing to determine one or more physiological parameters of a subject. A photoacoustic signal generated in response to a photonic signal may include multiple peaks as a result of multiple blood vessels and other structures below the surface of the skin of a subject. A photoacoustic system may identify a first and second peak in the photoacoustic signal and determine values from the peaks indicative of physiological parameters. Physiological parameters, such as venous oxygen saturation and arterial oxygen saturation, may be determined based on the values.

The present disclosure relates to determining physiological parameters, and more particularly relates to determining physiological parameters using multiple peak analysis in a photoacoustic system.

### SUMMARY

A physiological monitoring system may be configured to determine one or more physiological parameters of a subject based on multiple peaks in an acoustic pressure signal. The system may include a light source that provides a photonic signal to the subject. The light source may emit light of one, two, or more wavelengths of light. The system may also include a detector to detect an acoustic pressure signal from the subject. The acoustic pressure signal may be generated by the absorption of at least some of the photonic signal by the subject. The acoustic pressure signal may include different components corresponding to the different wavelengths of light provided by the light source.

A photoacoustic signal generated in response to a photonic signal may include multiple peaks. The peaks may correspond to components of the subject, such as blood vessels and other structures below the surface of the skin of the subject. The system may analyze the photoacoustic signal by identifying multiple peaks based on the signal. The peaks may be identified, for example, by using fixed or variable thresholds.

The system may determine one or more physiological parameters, such as oxygen saturation, the concentration of hemoglobin (e.g., oxygenated, deoxygenated, and/or total hemoglobin), or both for blood vessels (e.g., arterial and venous) based on peak information contained in the photoacoustic signal. The system may determine values indicative of a physiological parameter, where the values correspond to peaks in the photoacoustic signal. For example, a first value may be determined based on a first peak and a second value may be determined based on a second peak. The physiological parameter may be determined based on the determined values. For example, if multiple peaks are identified with a range of corresponding values indicative of oxygen saturation, the values can be analyzed to determine a desired physiological parameter. When arterial saturation is the desired physiological parameter, the highest value indicative of oxygen saturation may be selected and used to determine the physiological parameter. When venous saturation is the desired physiological parameter, the lowest value indicative of oxygen saturation may be selected and used to determine the physiological parameter.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative physiological monitoring system in accordance with some embodiments of the present disclosure;

FIG. 2 is a block diagram of the illustrative physiological monitoring system of FIG. 1 coupled to a subject in accordance with some embodiments of the present disclosure;

FIG. 3 is a block diagram of an illustrative signal processing system in accordance with some embodiments of the present disclosure;

FIG. 4 is an illustrative photoacoustic arrangement in accordance with some embodiments of the present disclosure;

FIG. 5 is a plot of an illustrative photoacoustic signal, including peaks corresponding to blood vessels in accordance with some embodiments of the present disclosure;

FIG. 6 is a flow diagram of illustrative steps for determining a physiological parameter in accordance with some embodiments of the present disclosure;

FIG. 7 is an illustrative plot of photoacoustic signals in accordance with some embodiments of the present disclosure;

FIG. 8 is an illustrative perspective view of a portion of the circulatory system in the neck of a subject in accordance with some embodiments of the present disclosure; and

FIG. 9 is another illustrative perspective view of a portion of the circulatory system in the neck of a subject in accordance with some embodiments of the present disclosure.

### DETAILED DESCRIPTION OF THE FIGURES

Photoacoustics (or “optoacoustics”) or “the photoacoustic effect” (or “optoacoustic effect”) refers to the phenomenon in which one or more wavelengths of light are presented to and absorbed by one or more constituents of an object, thereby causing an increase in kinetic energy of the one or more constituents, which causes an associated pressure response within the object. Particular modulations or pulsing of the incident light, along with measurements of the corresponding pressure response in, for example, tissue of the subject, may be used for physiological parameter determination, medical imaging, or both. For example, oxygen saturation and/or the concentration of a constituent such as hemoglobin (e.g., oxygenated, deoxygenated, and/or total hemoglobin), may be determined using photoacoustic analysis.

Hemoglobin is understood herein to be a complex protein carried in the bloodstream of a subject that is typically involved in transporting oxygen. Hemoglobin can carry oxygen by varying the oxidation state of an iron atom within the hemoglobin protein. Hemoglobin can be found in at least two states such as oxyhemoglobin and deoxyhemoglobin. Oxyhemoglobin is understood to represent the oxygenated state of hemoglobin. Oxyhemoglobin is involved in the process of transporting oxygen molecules from, for example, the lungs to various muscles, organs and other tissues of the subject. Deoxyhemoglobin is understood to be the deoxygenated state of hemoglobin, which is occurs, for example, after a molecule of oxyhemoglobin releases oxygen for delivery to a muscle, organ, or other tissue of the subject.

A photoacoustic system may include a photoacoustic sensor that is placed at a site on a subject, typically a cheek, tongue, temple, neck, palm, fingertip, toe, forehead or earlobe, or in the case of a neonate, across a foot. In some embodiments, the photoacoustic sensor can be placed anywhere where an artery or vessel is accessible noninvasively. The photoacoustic system may use a light source, and any suitable light guides (e.g., fiber optics), to pass light through the subject's tissue, or a combination of tissue thereof (e.g., organs) and an acoustic detector to sense the pressure response of the tissue induced by light absorption. Tissue may include muscle, fat, blood, blood vessels, and/or any other suitable tissue types. In some embodiments, the light source may be a laser or laser diode, operated in pulsed or continuous wave (CW) mode. In some embodiments, the acoustic detector may be an ultrasound detector, which may be suitable to detect pressure fluctuations arising from the constituent's absorption of the incident light of the light source.

In some embodiments, the light from the light source may be focused, shaped, or otherwise spatially modulated to illuminate a particular region of interest. In some arrangements, photoacoustic monitoring may allow relatively higher spatial resolution than line of sight optical techniques (e.g., path integrated absorption measurements). The enhanced spatial resolution of the photoacoustic technique may allow for imaging, scalar field mapping, and other spatially resolved results, in 1, 2, or 3 spatial dimensions. The acoustic response to the photonic excitation may radiate from the illuminated region of interest, and accordingly may be detected at multiple positions.

The photoacoustic system may measure the pressure response that is received at the acoustic sensor as a function of time. The photoacoustic system may also include sensors at multiple locations. A signal representing pressure versus time or a mathematical manipulation of this signal (e.g., a scaled version thereof, etc.) may be referred to as the photoacoustic signal. The photoacoustic signal may be derived from a detected acoustic pressure signal by selecting a suitable subset of points of an acoustic pressure signal. The photoacoustic signal may also be derived using an envelope technique on the absolute values of the acoustic pressure signal. The photoacoustic signal may be used to calculate any of a number of physiological parameters, including oxygen saturation and a concentration of a blood constituent (e.g., oxyhemoglobin), at a particular spatial location. In some embodiments, photoacoustic signals from multiple spatial locations may be used to construct an image (e.g., imaging blood vessels) or a scalar field (e.g., a hemoglobin concentration field). As used herein, blood vessels are understood to be the veins, arteries, and capillaries of a subject.

In some applications, the light passed through the tissue is selected to be of one or more wavelengths that are absorbed by the constituent in an amount representative of the amount of the constituent present in the tissue. The absorption of light passed through the tissue varies in accordance with the amount of the constituent in the tissue. For example, Red and/or infrared (IR) wavelengths may be used because highly oxygenated blood will absorb relatively less Red light and more IR light than blood with a lower oxygen saturation.

Any suitable light source may be used, and characteristics of the light provided by the light source may be controlled in any suitable manner. In some embodiments, a pulsed light source may be used to provide relatively short-duration pulses (e.g., nano-second pulses) of light to the region of interest. Accordingly, the use of a pulse light source may result in a relatively broadband acoustic response (e.g., depending on the pulse duration). The use of a pulsed light source will be referred to herein as the “Time Domain Photoacoustic” (TD-PA) technique. A convenient starting point for analyzing a TD-PA signal is given by Eq. 1:

p(z)=Γμₐφ(z) (1)

under conditions where the irradiation time is small compared to the characteristic thermal diffusion time determined by the properties of the specific tissue type. Referring to Eq. 1, p(z) is the photoacoustic signal (indicative of the maximum induced pressure rise, derived from an acoustic signal) at spatial location z indicative of acoustic pressure, Γ is the dimensionless Grüneisen parameter of the tissue, μₐ is the effective absorption coefficient of the tissue (or constituent thereof) to the incident light, and Φ(z) is the optical fluence at spatial location z. The Grüneisen parameter is a dimensionless description of thermoelastic effects, and may be illustratively formulated by Eq. 2:

$$Γ = \frac{β {c}_{a}^{2}}{{C}_{P}} \qquad ( 2 )$$

where cₐ is the speed of sound in the tissue, β is the isobaric volume thermal expansion coefficient, and C_{P} is the specific heat at constant pressure. In some circumstances, the optical fluence, at spatial location z (within the subject's tissue) of interest may be dependent upon the light source, the location itself (e.g., the depth), and optical properties (e.g., scattering coefficient, absorption coefficient, or other properties) along the optical path. For example, Eq. 3 provides an illustrative expression for the attenuated optical fluence at a depth z:

φ(z)=φ₀e^{-μ}^{_{eff}}^{z} (3)

where φ₀ is the optical fluence from the light source incident at the tissue surface, z is the path length (i.e., the depth into the tissue in this example), and μ_{eff} is an effective attenuation coefficient of the tissue along the path length in the tissue in this example.

In some embodiments, a more detailed expression or model may be used rather than the illustrative expression of Eq. 3. In some embodiments, the actual pressure encountered by an acoustic detector may be proportional to Eq. 1, as the focal distance and solid angle (e.g., face area) of the detector may affect the actual measured photoacoustic signal. In some embodiments, an ultrasound detector positioned relatively farther away from the region of interest, will encounter a relatively smaller acoustic pressure. For example, the peak acoustic pressure signal received at a circular area A_{d} positioned at a distance R from the illuminated region of interest may be given by Eq. 4:

p_{d}=p(z)f(rₛ,R,A_{d}) (4)

where rₛ is the radius of the illuminated region of interest (and typically rₛ<R), and p(z) is given by Eq. 1. In some embodiments, the detected acoustic pressure amplitude may decrease as the distance R increases (e.g., for a spherical acoustic wave).

In some embodiments, a modulated CW light source may be used to provide a photonic excitation of a tissue constituent to cause a photoacoustic response in the tissue. The CW light source may be intensity modulated at one or more characteristic frequencies. The use of a CW light source, intensity modulated at one or more frequencies, will be referred to herein as the “Frequency Domain Photoacoustic” (FD-PA) technique. Although the FD-PA technique may include using frequency domain analysis, the technique may use time domain analysis, wavelet domain analysis, or any other suitable analysis, or any combination thereof. Accordingly, the term “frequency domain” as used in “FD-PA” refers to the frequency modulation of the photonic signal, and not to the type of analysis used to process the photoacoustic response.

Under some conditions, the acoustic pressure p(R,t) at detector position R at time t, may be shown illustratively by Eq. 5:

$$p ( R , t ) \sim \frac{{p}_{0} ( {r}_{0} , ω )}{R} {e}^{- iω ( t - τ )} \qquad ( 5 )$$

where r₀ is the position of the illuminated region of interest, ω is the angular frequency of the acoustic wave (caused by modulation of the photonic signal at frequency ω), R is the distance between the illuminated region of interest and the detector, and τ is the travel time delay of the wave equal to R/cₐ, where cₐ is the speed of sound in the tissue. The FD-PA spectrum p₀(r₀,ω) of acoustic waves is shown illustratively by Eq. 6:

$${p}_{0} ( {r}_{0} , ω ) = \frac{{Γμ}_{a} φ ( {r}_{0} )}{2 ( {μ}_{a} {c}_{a} - iω )} \qquad ( 6 )$$

where μₐcₐ represents a characteristic frequency (and corresponding time scale) of the tissue.

In some embodiments, a FD-PA system may temporally vary the characteristic modulation frequency of the CW light source, and accordingly the characteristic frequency of the associated acoustic response. For example, the FD-PA system may use linear frequency modulation (LFM), either increasing or decreasing with time, which is sometimes referred to as “chirp” signal modulation. Shown in Eq. 7 is an illustrative expression for a sinusoidal chirp signal r(t):

$$r ( t ) = cos ( t ( {ω}_{0} + \frac{b}{2} t ) ) \qquad ( 7 )$$

where ω₀ is a starting angular frequency, and b is the angular frequency scan rate. Any suitable range of frequencies (and corresponding angular frequencies) may be used for modulation such as, for example, 1-5 MHz, 200-800 kHz, or other suitable range, in accordance with the present disclosure. In some embodiments, signals having a characteristic frequency that changes as a nonlinear function of time may be used. Any suitable technique, or combination of techniques thereof, may be used to analyze a FD acoustic pressure signal. Two such exemplary techniques, a correlation technique and a heterodyne mixing technique, will be discussed below as illustrative examples.

In some embodiments, the correlation technique may be used to determine the travel time delay of the FD-PA signal. In some embodiments, a matched filtering technique may be used to process a photoacoustic signal. As shown in Eq. 8:

$${B}_{s} ( t - τ ) = \frac{1}{2 π} {\int }_{- ∞}^{∞} H ( ω ) S ( ω ) {e}^{iω t} d ω \qquad ( 8 )$$

Fourier transforms (and inverse transforms) are used to calculate the filter output Bₛ(t−T), in which H(ω) is the filter frequency response, S(ω) is the Fourier transform of the photoacoustic signal s(t), and T is the phase difference between the filter and signal. In some circumstances, the filter output of expression of Eq. 8 may be equivalent to an autocorrelation function. Shown in Eq. 9:

$$S ( ω ) = \frac{1}{2 π} {\int }_{- ∞}^{∞} s ( t ) {e}^{- iω t} d t \qquad ( 9 )$$

is an expression for computing the Fourier transform S(ω) of the photoacoustic signal s(t). Shown in Eq. 10:

H(ω)=S*(ω)e^{-iωτ} (10)

is an expression for computing the filter frequency response H(ω) based on the Fourier transform of the photoacoustic signal s(t), in which S*(ω) is the complex conjugate of S(ω). It can be observed that the filter frequency response of Eq. 10 requires the frequency character of the photoacoustic signal be known beforehand to determine the frequency response of the filter. In some embodiments, as shown by Eq. 11:

$$B ( t ) = {\int }_{- ∞}^{∞} r ( {t}^{′} ) s ( t + {t}^{′} ) {d}^{′} \qquad ( 11 )$$

the known modulation signal r(t) may be used for generating a cross-correlation with the photoacoustic signal. The cross-correlation output B(t) of Eq. 11 is expected to exhibit a peak at a time t equal to the acoustic signal travel time τ. Assuming that the temperature response and resulting acoustic response follow the illumination modulation (e.g., are coherent), Eq. 11 may allow calculation of the time delay, depth information, or both.

In some embodiments, the heterodyne mixing technique may be used to determine the travel time delay of the FD-PA signal. The FD-PA signal, as described above, may have similar frequency character as the modulation signal (e.g., coherence), albeit shifted in time due to the travel time of the acoustic signal. For example, a chirp modulation signal, such as r(t) of Eq. 7, may be used to modulate a CW light source. Heterodyne mixing uses the trigonometric identity of the following Eq. 12:

$$cos ( A ) cos ( B ) = \frac{1}{2} [ cos ( A - B ) - cos ( A + B ) ] \qquad ( 12 )$$

which shows that two signals may be combined by multiplication to give periodic signals at two distinct frequencies (i.e., the sum and the difference of the original frequencies). If the result is passed through a low-pass filter to remove the higher frequency term (i.e., the sum), the resulting filtered, frequency shifted signal may be analyzed. For example, Eq. 13 shows a heterodyne signal L(t):

$$L ( t ) = 〈 r ( t ) s ( t ) 〉 ≅ 〈 Kr ( t ) r ( t - \frac{R}{{c}_{a}} ) 〉 = \frac{1}{2} K cos ( \frac{R}{{c}_{a}} bt + θ ) \qquad ( 13 )$$

calculated by low-pass filtering (shown by angle brackets) the product of modulation signal r(t) and photoacoustic signal s(t). If the photoacoustic signal is assumed to be equivalent to the modulation signal, with a time lag R/cₐ due to travel time of the acoustic wave and amplitude scaling K, then a convenient approximation of Eq. 13 may be made, giving the rightmost expression of Eq. 13. Analysis of the rightmost expression of Eq. 13 may provide depth information, travel time, or both. For example, a fast Fourier transform (FFT) may be performed on the heterodyne signal, and the frequency associated with the highest peak may be considered equivalent to time lag Rb/cₐ. Assuming that the frequency scan rate b and the speed of sound cₐ are known, the depth R may be estimated.

Venous oxygen saturation is a physiological parameter that may be used to assess a subject's condition. For example, venous oxygen saturation is one of the key parameters that physicians use to assess the status of critically ill subjects. Invasive techniques for determining venous oxygen saturation may cause complications. Accordingly, a non-invasive technique for determining venous oxygen saturation (e.g., based on photoacoustic measurements) may be highly desirable. In some embodiments, a photoacoustic measurement of the jugular vein may allow for a rapid, non-invasive, beneficial assessment of a subject's health.

In some embodiments, a photoacoustic measurement may be carried out such that signals from multiple structures, blood vessels, organs, or tissues are detected. For example, a photoacoustic detector located near the neck of a subject may detect an acoustic signal from the skin, external jugular vein, internal jugular vein, external carotid artery, sternocleidomastoid muscle, other internal and external signals, or any combination thereof. In a time or distance resolved photoacoustic measurement, correlating measured signal peaks with target area structures may enable the determination of physiological parameters. In some embodiments, the peaks may be differentiated and may be correlated to physiological parameters based on their amplitudes.

In some embodiments, the oxygen saturation of a peak in an acoustic signal may be determined using one or more of the techniques described herein. The peak with the smallest saturation number may correspond to the venous oxygen saturation, as venous blood is understood to contain a lower proportion of oxyhemoglobin and the higher percentage of deoxyhemoglobin than arterial blood. Similarly, the peak with the largest saturation number may correspond to the arterial oxygen saturation. In some embodiments, the concentration of oxyhemoglobin and deoxyhemoglobin of a peak in an acoustic signal may be determined using one or more of the techniques described herein.

In some embodiments, multiple peaks in the acoustic signal may be detected. The peaks may be detected in an acoustic signal corresponding to a single wavelength of light or in acoustic signals corresponding to multiple wavelengths of light. The acoustic peaks may be processed using processing equipment to determine physiological parameters from the peaks. Relative and absolution comparisons may be used to determine which peak corresponds to and therefore contains the desired physiological information.

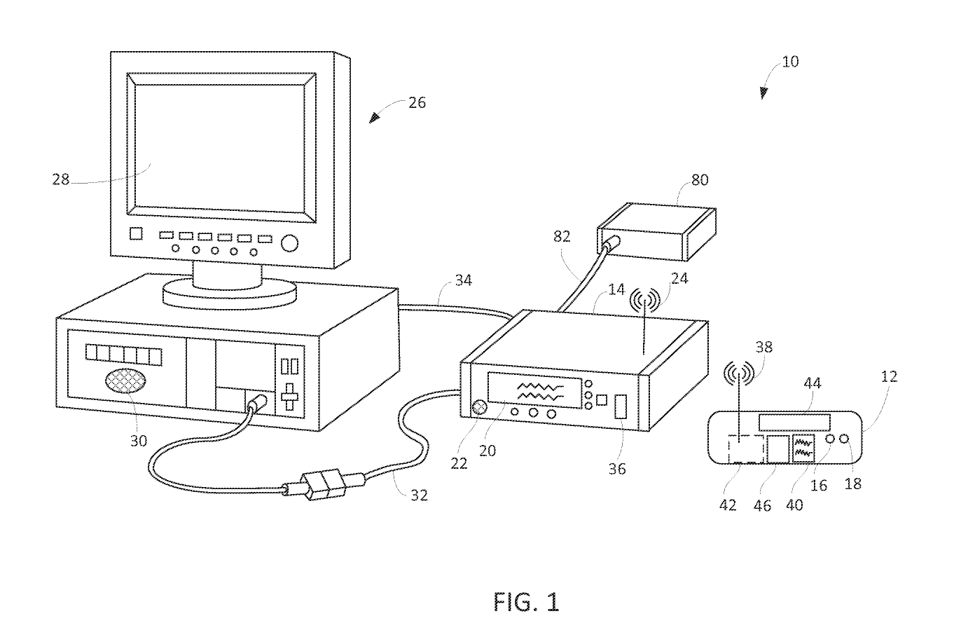

The following description and accompanying FIGS. 1-9 provide additional details and features of some embodiments of multiple peak analysis in a photoacoustic system.

FIG. 1 shows an illustrative physiological monitoring system in accordance with some embodiments of the present disclosure. System 10 may include sensor unit 12 and monitor 14. In some embodiments, sensor unit 12 may be part of a photoacoustic monitor or imaging system. Sensor unit 12 may include a light source 16 for emitting light at one or more wavelengths into a subject's tissue, which may but need not correspond to visible light, into a subject's tissue. Light source 16 may provide a photonic signal including any suitable electromagnetic radiation such as, for example, a radio wave, a microwave wave, an infrared wave, a visible light wave, ultraviolet wave, any other suitable light wave, or any combination thereof. A detector 18 may also be provided in sensor unit 12 for detecting the acoustic (e.g., ultrasound) response that travels through the subject's tissue. Any suitable physical configuration of light source 16 and detector 18 may be used. In some embodiments, sensor unit 12 may include multiple light sources and/or acoustic detectors, which may be spaced apart.

System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12 (e.g., a photoplethysmograph sensor). Multiple sensor units may be capable of being positioned at two different locations on a subject's body.

In some embodiments, system 10 may include two or more sensors forming a sensor array in lieu of either or both of the sensor units. In some embodiments, a sensor array may include multiple light sources, detectors, or both. It will be understood that any type of sensor, including any type of physiological sensor, may be used in one or more sensor units in accordance with the systems and techniques disclosed herein. It will be understood that any number of sensors measuring any number of physiological signals may be used to determine physiological information in accordance with the techniques described herein.

In some embodiments, the sensor may be wirelessly connected to monitor 14 (e.g., via wireless transceivers 38 and 24) and include its own battery or similar power source 44. In some embodiments, sensor unit 12 may draw its power from monitor 14 and be communicate with monitor 14 via a physical connection such as a wired connection (not shown). Sensor unit 12, monitor 14, or both, may be configured to calculate physiological parameters based at least in part on data relating to light emission and acoustic detection received at one or more sensor units such as sensor unit 12. For example, sensor unit 12, monitor 14, or both, may be configured to determine blood oxygen saturation (e.g., arterial, venous, or both), pulse rate, blood pressure, hemoglobin concentration (e.g., oxygenated, deoxygenated, or total), any other suitable physiological parameters, or any combination thereof. In some embodiments, some or all calculations may be performed on sensor unit 12 (i.e., using processing equipment 42) or an intermediate device and the result of the calculations may be passed to monitor 14. Further, monitor 14 may include monitor display 20 configured to display the physiological parameters or other information about the system. Sensor unit 12 may also include a sensor display 40 configured to display the physiological parameters or other information about the system and a user interface 46. In an exemplary embodiment, processing equipment 42 may be configured to operate light source 16 and detector 18 to generate and process acoustic signals, communicate with display sensor 40 to display values such as signal quality and power levels, receive signals from user input 46, and control wireless transceiver 38 to communicate data (e.g., acoustic output signals) with monitor 14.

In the embodiment shown, monitor 14 may also include speaker 22 to provide an audible sound that may be used in various other embodiments, such as for example, sounding an audible alarm in the event that a subject's physiological parameters are not within a predefined normal range. In another embodiment, sensor unit 12 may communicate such information to the user, e.g., using sensor display 40, an audible source such as a speaker, vibration, tactile, or any other way for communicating a status to a user, such as for example, in the event that a subject's physiological parameters are not within a predefined normal range.

In some embodiments, sensor unit 12 may be communicatively coupled to monitor 14 via a wireless system, utilizing antenna 38 of sensor unit 12 and antenna 24 of monitor 14. Antenna 38 may be external or internal to sensor unit 12, and capable of transmitting signals, receiving signals, or both transmitting and receiving signals, via amplitude modulated RF, frequency modulated RF, Bluetooth, IEEE 802.11, WiFi, WiMax, cable, satellite, infrared, any other suitable transmission scheme, or any combination thereof. Communication between the sensor unit 12 and monitor 14 may also be carried over a cable (not shown) to an input 36 of monitor 14, or to a multi-parameter physiological monitor 26 (described below). The cable may include electronic conductors (e.g., wires for transmitting electronic signals from detector 18, or a partially or fully processed signal from sensor unit 12), optical fibers (e.g., multi-mode or single-mode fibers for transmitting emitted light from light source 16), any other suitable components, any suitable insulation or sheathing, or any combination thereof. Monitor 14 may include a sensor interface configured to receive physiological signals from sensor unit 12, provide signals and power to sensor unit 12, transfer data specific to the subject, general to the physiological parameter being measured, or both, or otherwise communicate with sensor unit 12. The sensor interface may include any suitable hardware, software, or both, which may allow communication between monitor 14 and sensor unit 12.

In the illustrated embodiment, system 10 includes multi-parameter physiological monitor 26. The monitor 26 may include a cathode ray tube display, a flat panel display (as shown) such as a liquid crystal display (LCD) or a plasma display, or may include any other type of monitor now known or later developed. Multi-parameter physiological monitor 26 may be configured to calculate physiological parameters and to provide a multi-parameter physiological monitor display 28 for information from sensor unit 12, monitor 14, or both, and from other medical monitoring devices or systems (not shown). For example, multi-parameter physiological monitor 26 may be configured to display an estimate of, for example, a subject's blood oxygen saturation, blood pressure, hemoglobin concentration, and/or pulse rate generated by sensor unit 12 or monitor 14. Multi-parameter physiological monitor 26 may include a speaker 30.

Monitor 14 may be communicatively coupled to multi-parameter physiological monitor 26 via a cable 32 or 34 that is coupled to a sensor input port or a digital communications port, respectively and/or may communicate wirelessly (not shown). The multi-parameter physiological monitor 26 may also be communicatively coupled to sensor unit 12 with or without the presence of monitor 14. Sensor unit 12 may be coupled to the multi-parameter physiological monitor 26 by a wireless connection using wireless transceiver 38 and a transceiver (not shown) on multi-parameter physiological monitor 26, or by a cable (not shown). In addition, sensor unit 12, monitor 14, or multi-parameter physiological monitor 26 may be coupled to a network to enable the sharing of information with servers or other workstations (not shown). In some embodiments this network may be a local area network, which may be further coupled through the internet or other wide area network for remote monitoring. Sensor unit 12, monitor 14 and multi-parameter physiological monitor 26 may be powered by a battery (not shown) or by a conventional power source such as a wall outlet.

Calibration device 80, which may be powered by monitor 14, a battery, or by a conventional power source such as a wall outlet, may include any suitable calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

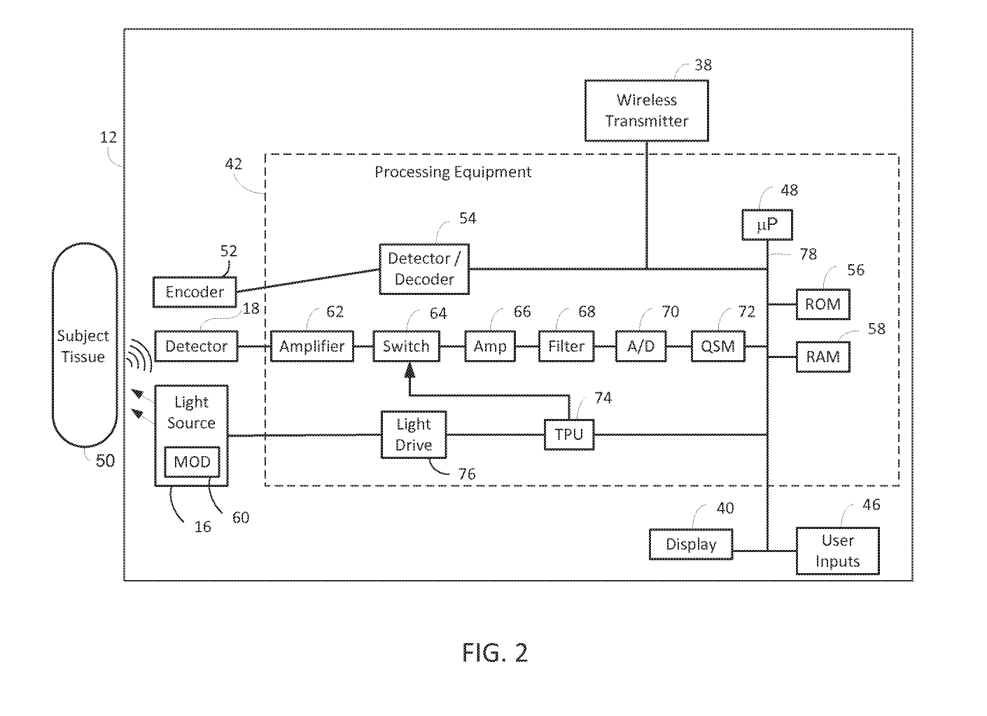

FIG. 2 is a block diagram of the illustrative physiological monitoring system of FIG. 1 coupled to a subject in accordance with some embodiments of the present disclosure. Physiological monitoring system 10 of FIG. 1 may be coupled to a subject's tissue 50 in accordance with an embodiment. Certain illustrative components of sensor unit 12 of FIG. 1 and monitor 14 of FIG. 1 are illustrated in FIG. 2. It will be understood that processing equipment 42 may be included fully or partially included in monitor 14 of FIG. 1, in or fully or partially in sensor unit 12, fully or partially in multi-parameter physiological monitor 26, in any other suitable arrangement, or any combination thereof. It will be understood that any displayed information may be displayed on sensor display 40, monitor display 20, multi-parameter physiological monitor display 28, other suitable display, or any combination thereof.

Sensor unit 12 may include light source 16, detector 18, and encoder 52. In some embodiments, light source 16 may be configured to emit one or more wavelengths of light (e.g., visible, infrared) into a subject's tissue 50. Hence, light source 16 may provide red light, IR light, any other suitable light, or any combination thereof, that may be used to calculate the subject's physiological parameters. In some embodiments, the red wavelength may be between about 600 nm and about 700 nm, and the IR wavelength may be between about 800 nm and about 1000 nm. In embodiments where a sensor array is used in place of a single sensor, each sensor may be configured to provide light of a single wavelength. For example, a first sensor may emit only a Red light while a second may emit only an IR light. In a further example, the wavelengths of light used may be selected based on the specific location of the sensor.

It will be understood that, as used herein, the term “light” may refer to energy produced by electromagnetic radiation sources. Light may be of any suitable wavelength and intensity, and modulations thereof, in any suitable shape and direction. Detector 18 may be chosen to be specifically sensitive to the acoustic response of the subject's tissue arising from use of light source 16. It will also be understood that, as used herein, the “acoustic response” shall refer to pressure and changes thereof caused by a thermal response (e.g., expansion and contraction) of tissue to light absorption by the tissue or constituent thereof.

In some embodiments, detector 18 may be configured to detect the acoustic response of tissue to the photonic excitation caused by the light source. In some embodiments, detector 18 may be a piezoelectric transducer which may detect force and pressure and output an electrical signal via the piezoelectric effect. In some embodiments, detector 18 may be a Faby-Pérot interferometer, or etalon. For example, a thin film (e.g., composed of a polymer) may be irradiated with reference light, which may be internally reflected by the film. Pressure fluctuations may modulate the film thickness, thus causing changes in the reference light reflection which may be measured and correlated with the acoustic pressure. In some embodiments, detector 18 may be configured or otherwise tuned to detect acoustic response in a particular frequency range. Detector 18 may convert the acoustic pressure signal into an electrical signal (e.g., using a piezoelectric material, photodetector of a Faby-Pérot interferometer, or other suitable device). After converting the received acoustic pressure signal to an electrical optical, and/or wireless signal, detector 18 may send the signal to processing equipment 42, where physiological parameters may be calculated based on the photoacoustic activity within the subject's tissue 50. The signal outputted from detector 18 and/or a pre-processed signal derived thereof, will be referred to herein as a photoacoustic signal.

In some embodiments, encoder 52 may contain information about sensor unit 12, such as what type of sensor it is (e.g., where the sensor is intended to be placed on a subject), the wavelength(s) of light emitted by light source 16, the intensity of light emitted by light source 16 (e.g., output wattage or Joules), the mode of light source 16 (e.g., pulsed versus CW), any other suitable information, or any combination thereof. This information may be used by processing equipment 42 to select appropriate algorithms, lookup tables, and/or calibration coefficients stored in processing equipment 42 for calculating the subject's physiological parameters.

Encoder 52 may contain information specific to subject's tissue 50, such as, for example, the subject's age, weight, and diagnosis. This information about a subject's characteristics may allow processing equipment 42 to determine, for example, subject-specific threshold ranges in which the subject's physiological parameter measurements should fall and to enable or disable additional physiological parameter algorithms. Encoder 52 may, for instance, be a coded resistor that stores values corresponding to the type of sensor unit 12 or the type of each sensor in the sensor array, the wavelengths of light emitted by light source 16 on each sensor of the sensor array, and/or the subject's characteristics. In some embodiments, encoder 52 may include a memory on which one or more of the following information may be stored for communication to processing equipment 42: the type of the sensor unit 12; the wavelengths of light emitted by light source 16; the particular acoustic range that each sensor in the sensor array is monitoring; the particular acoustic spectral characteristics of a detector; a signal threshold for each sensor in the sensor array; any other suitable information; or any combination thereof.

In some embodiments, signals from detector 18 and encoder 52 may be transmitted to processing equipment 42. In the embodiment shown, processing equipment 42 may include a general-purpose microprocessor 48 connected to an internal bus 78. Microprocessor 48 may be adapted to execute software, which may include an operating system and one or more applications, as part of performing the functions described herein. Also connected to bus 78 may be a read-only memory (ROM) 56, a random access memory (RAM) 58, user inputs 46, sensor display 40, and speaker 22 of FIG. 1.

RAM 58 and ROM 56 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules, or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, time processing unit (TPU) 74 may provide timing control signals to light drive circuitry 76, which may control the activation of light source 16. For example, TPU 74 may control pulse timing (e.g., pulse duration and inter-pulse interval) for TD-PA monitoring system. TPU 74 may also control the gating-in of signals from detector 18 through amplifier 62 and switching circuit 64. The received signal from detector 18 may be passed through amplifier 66, low pass filter 68, and analog-to-digital converter 70. The digital data may then be stored in a queued serial module (QSM) 72 (or buffer) for later downloading to RAM 58 as QSM 72 is filled. In some embodiments, there may be multiple separate parallel paths having components equivalent to amplifier 62, filter 68, and/or analog-to-digital converter 70 for multiple light wavelengths or spectra received. Any suitable combination of components (e.g., microprocessor 48, RAM 58, analog-to-digital converter 70, any other suitable component shown or not shown in FIG. 2) coupled by bus 78 or otherwise coupled (e.g., via an external bus), may be referred to as “processing equipment.”

In the embodiment shown, light source 16 may include modulator 60, in order to, for example, perform FD-PA analysis. Modulator 60 may be configured to provide intensity modulation, spatial modulation, any other suitable optical signal modulations, or any combination thereof. For example, light source 16 may be a CW light source, and modulator 60 may provide intensity modulation of the CW light source such as using a linear sweep modulation. In some embodiments, modulator 60 may be included in light drive 60, or other suitable components of physiological monitoring system 10, or any combination thereof.

In some embodiments, microprocessor 48 may determine the subject's physiological parameters, such as SpO₂, SvO₂, total hemoglobin concentration (t_{HB}), oxyhemoglobin concentration, deoxyhemoglobin concentration, and/or pulse rate, using various algorithms and/or look-up tables based on the value of the received signals and/or data corresponding to the acoustic response received by detector 18. Signals corresponding to information about subject 50, and particularly about the acoustic signals emanating from a subject's tissue over time, may be transmitted from encoder 52 to decoder 54. These signals may include, for example, encoded information relating to subject characteristics. Decoder 54 may translate these signals to enable the microprocessor to determine the thresholds based at least in part on algorithms or look-up tables stored in ROM 56. In some embodiments, user inputs 46 may be used enter information, select one or more options, provide a response, input settings, any other suitable inputting function, or any combination thereof. User inputs 46 may be used to enter information about the subject, such as, for example, age, weight, height, diagnosis, medications, treatments, and so forth. In some embodiments, sensor display 40 may exhibit a list of values, which may generally apply to the subject, such as, for example, age ranges or medication families, which the user may select using user inputs 46.

The acoustic signal attenuated by the tissue of subject 50 can be degraded by noise, among other sources. Movement of the subject may also introduce noise and affect the signal. For example, the contact between the detector and the skin, or the light source and the skin, can be temporarily disrupted when movement causes either to move away from the skin. Another potential source of noise is electromagnetic coupling from other electronic instruments.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Processing sensor signals may involve operations that reduce the amount of noise present in the signals, control the amount of noise present in the signal, or otherwise identify noise components in order to prevent them from affecting measurements of physiological parameters derived from the sensor signals.

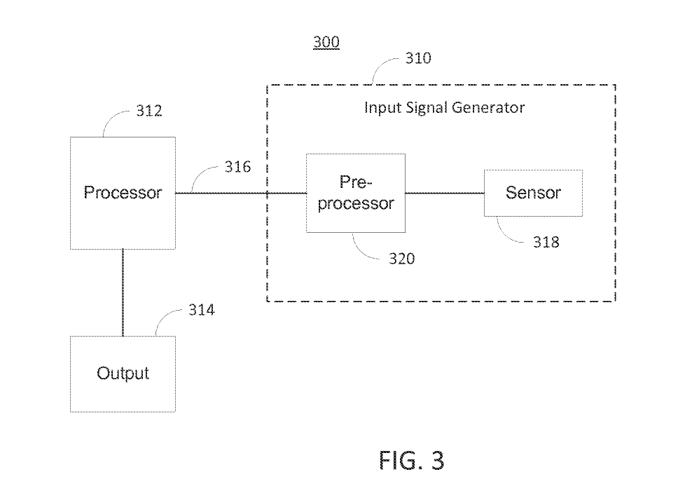

FIG. 3 is a block diagram of an illustrative signal processing system in accordance with some embodiments of the present disclosure. Signal processing system 300 may implement the signal processing techniques described herein. In some embodiments, signal processing system 300 may be included in a physiological monitoring system (e.g., physiological monitoring system 10 of FIGS. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be a photoacoustic module and input signal 316 may be a photoacoustic signal. In some embodiments, pre-processor 320 may be any suitable signal processing device and input signal 316 may include one or more photoacoustic signals and one or more other physiological signals, such as a photoplethysmograph signal. It will be understood that input signal generator 310 may include any suitable signal source, signal generating data, signal generating equipment, or any combination thereof to produce input signal 316. Input signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, input signal 316 may be coupled to processor 312. Processor 312 may be any suitable software, firmware, hardware, or combination thereof for processing input signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may perform time domain calculations, spectral domain calculations, time-spectral transformations (e.g., fast Fourier transforms, inverse fast Fourier transforms), any other suitable calculations, or any combination thereof. Processor 312 may perform any suitable signal processing of input signal 316 to filter input signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the subject. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize input signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. In some embodiments, processor 312 may store physiological measurements or previously received data from signal 316 in a memory device for later retrieval. In some embodiments, processor 312 may store calculated values, such as pulse rate, blood pressure, blood oxygen saturation (e.g., arterial, venous, or both), hemoglobin concentration (e.g., oxygenated, deoxygenated, or total), any other suitable calculated values, or combinations thereof, in a memory device for later retrieval.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIG. 1), in which, for example, input signal generator 310 may be implemented as part of sensor unit 12 (FIGS. 1 and 2), monitor 14 (FIG. 1), and processing equipment 42 (FIG. 2), and processor 312 may be implemented as part of monitor 14 (FIG. 1) and processing equipment 42 (FIG. 2). In some embodiments, portions of system 300 may be configured to be portable. For example, all or part of system 300 may be embedded in a small, compact object carried with or attached to the subject (e.g., a watch, other piece of jewelry, or a smart phone). In some embodiments, a wireless transceiver (not shown) may also be included in system 300 to enable wireless communication with other components of system 10 (FIG. 1). As such, system 10 (FIG. 1) may be part of a fully portable and continuous physiological monitoring solution. In some embodiments, a wireless transceiver (not shown) may also be included in system 300 to enable wireless communication with other components of system 10. For example, pre-processor 320 may output signal 316 (e.g., which may be a pre-processed photoacoustic signal) over BLUETOOTH, IEEE 802.11, WiFi, WiMax, cable, satellite, Infrared, any other suitable transmission scheme, or any combination thereof. In some embodiments, a wireless transmission scheme may be used between any communicating components of system 300.

It will also be understood that while some of the equations referenced herein are continuous functions, the processing equipment may be configured to use digital or discrete forms of the equations in processing the acquired photoacoustic signals.

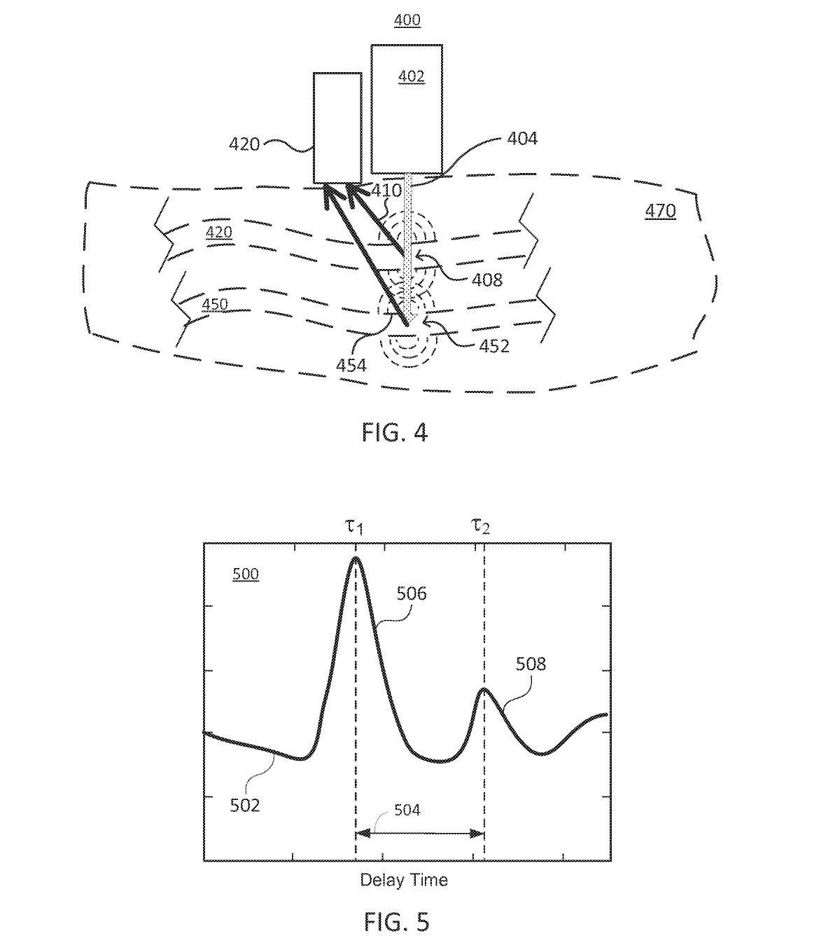

FIG. 4 is an illustrative photoacoustic arrangement in accordance with some embodiments of the present disclosure. The arrangement 400 may include light source 402, controlled by a suitable light drive (e.g., a light drive of system 300 of FIG. 3 or system 10 of FIG. 1, although not shown in FIG. 4). Light source 402 may provide photonic signal 404 to subject tissue 470 including blood vessel 420 and blood vessel 450. Photonic signal 404 may be attenuated along its path length by subject tissue 470 prior to reaching target area 408 for blood vessel 420 and target area 452 for blood vessel 450. It will be understood that photonic signal 404 may scatter in subject 450 and need not travel in a well-formed beam as illustrated. Also, photonic signal 404 may generally travel through and beyond blood vessels 420 and 450. A constituent of the blood in blood vessels 420 and 450 such as, for example, hemoglobin, may absorb at least some of photonic signal 404. Accordingly, the blood may exhibit an acoustic pressure response via the photoacoustic effect, which may act on the surrounding tissues of blood vessels 420 and 450. Photoacoustic signal 410 may be generated near target area 408 of blood vessel 420 and may travel through subject 470 in all directions. Photoacoustic signal 454 may be generated near target area 452 of blood vessel 450 and may travel through the subject in all directions. Acoustic detector 420 (i.e., a photoacoustic detector) may detect acoustic pressure signals corresponding to photoacoustic signals 410 and 454. Acoustic detector 420 may output a signal for further processing. Because the path length between target area 408 and acoustic detector 420 is shorter than the pathlength between target area 452 and acoustic detector 420, it may be expected that acoustic detector 420 may receive acoustic pressure signals from target area 408 before receiving acoustic pressure signals from target area 452. In some embodiments, the relatively shorter path length between target area 408 and acoustic detector 420 and relatively longer path length between target area 452 and acoustic detector 402 may result in acoustic pressure signal 410 being relatively less attenuated and acoustic pressure signal 454 being relatively more attenuated.

FIG. 5 is a plot of an illustrative photoacoustic signal, including peaks corresponding to blood vessels in accordance with some embodiments of the present disclosure. The photoacoustic signal of FIG. 5 may have been generated, for example, based on an envelope detection performed on a photoacoustic sensor signal. The abscissa of plot 500 is presented in units proportional to time (e.g., delay time relative to a light pulse), while the ordinate of plot 500 is presented in arbitrary units of signal intensity. The system may receive or derive photoacoustic signal 502. At least a portion of photoacoustic signal 502 may relate to the acoustic pressure response of blood within a blood vessel. Photoacoustic signal 502 may display a first peak 506 at time τ₁ and a second peak 508 at time Σ₂. The first peak corresponds to a first blood vessel. The second peak corresponds to a second blood vessel. In some embodiments, two or more peaks, in part overlapping, may be identified relating to blood vessels or other structure. Time difference 504 between τ₁ and τ₂ indicates the relative difference in delay time between photoacoustic signals from the two vessels. The signal intensity may correspond to the absorption of particular constituent(s) of the target area or areas. In some embodiments, analysis of two or more peaks may allow the determination of one or more physiological parameters.

In some embodiments, the peaks of FIG. 5 may represent a photoacoustic signal detected by photoacoustic detector 420 of FIG. 4 in the arrangement shown in FIG. 4. For example, peak 506 may correspond to the acoustic signal 410 generated at target area 408 of vessel 420 and peak 508 may correspond to the acoustic signal 454 generated at target area 452 of vessel 450.

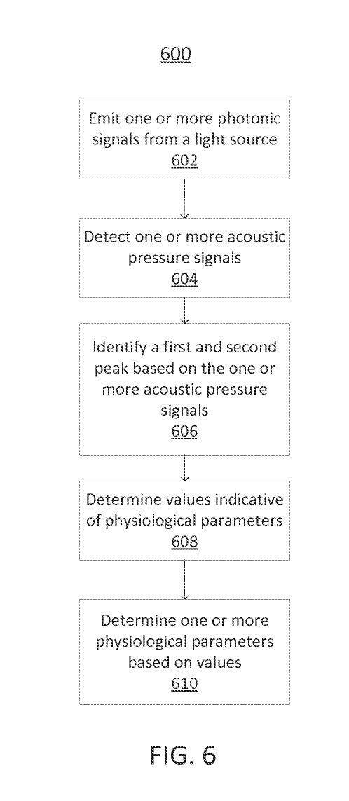

FIG. 6 is a flow diagram of illustrative steps for determining a physiological parameter in accordance with some embodiments of the present disclosure. In step 602, the system may emit one or more photonic signals from one or more light sources. The one or more light sources may, for example, be light source 16 of FIG. 1. In some embodiments, the system may include one or more light sources configured to emit particular wavelengths of light including red light, IR light, any other suitable light, or any combination thereof. The system may use particular wavelengths of light to determine physiological parameters of a subject. In some embodiments, the photonic signal may include a first light substantially centered at a first wavelength. The photonic signal may include a second light substantially centered at a second wavelength. The system may emit the first and second wavelengths of light concurrently, alternatingly, in any other suitable arrangement, or any combination thereof. In some embodiments, the system may emit a continuous wave photonic signal. The continuous wave light source may include frequency, time, or phase modulated signals.

It will be understood that the one or more light sources may not be purely monochromatic. For example, light referred to herein as 700 nm may be a Gaussian, Lorentzian, other distribution, or any combination thereof, centered at 700 nm. The distribution may have a relatively sharp form, such that, for example, 90% of a light source centered at 700 nm is between 695 nm and 705 nm. The light may be generated using a substantially single color lamp such as a diode emitter, laser diode emitter, or laser. The light may be generated using a continuous or multi-peak emitting light such as a tungsten filament lamp, Xe discharge lamp, Hg discharge lamp, other suitable light source, or any combination thereof. The system may filter and condition the light using high-pass filters, low-pass filters, band-pass filters, band-stop filters, prisms, diffraction gratings, mirrors, lenses, other suitable light conditioning devices, or any combination thereof.

In step 604, the system may detect an acoustic pressure signal generated in the tissue of a subject in response to the one or more photonic signals. This acoustic pressure signal may be detected using an acoustic detector. The acoustic detector may, for example, be detector 18 of FIG. 1. The acoustic pressure signal may include a pressure signal generated as a result of the photoacoustic effect, as described above. The acoustic detector may include an ultrasonic detector or microphone capable of detecting an acoustic pressure signal. The corresponding photoacoustic signal may include one or more components corresponding to the one or more photonic signals. It will be understood that components may also be referred to herein as separate signals. In some embodiments, the photoacoustic signal may be a processed version of the acoustic detector signal. For example, the photoacoustic signal may be derived based on an envelope detection performed on the acoustic detector signal.

In some embodiments, a first wavelength photonic signal may generate a first photoacoustic signal and a second wavelength photonic signal may generate a second photoacoustic signal. These measurements may be made concurrently or consecutively in an alternating fashion. Concurrently measured signals at multiple wavelengths may be measured at spatially separated locations. Emitting photonic signals and detecting acoustic pressure signals may be repeated multiple times. For example, a measurement as described herein may be carried out 5 times at a first wavelength and 5 times at a second wavelength. The 10 measurements may take place within one cardiac pulse cycle or over several cardiac pulse cycles such that the physiological parameters remain relatively constant. In some embodiments, the system may average measurements over, for example, seconds, minutes or hours, to improve the signal-to-noise ratio, determine a baseline, to monitor changes over time, for any other suitable reason, or any combination thereof. In another example, the system may overlay photoacoustic signals generated by multiple wavelengths to compare the signals. The multiple photoacoustic signals may be aligned to account for small shifts in time, distance, other values, or any combination thereof. In some embodiments, the system may align multiple photoacoustic signals in time with respect to the emission timing of a photonic signal from the light source. In some embodiments, the system may determine and use the center of a particular peak to align multiple signals. It will be understood that the aforementioned alignment methods are provided as examples, and other methods may be employed as well. It will also be understood that any combination of the aforementioned and other methods may be employed. It will also be understood that instead of aligning the photoacoustic signals, corresponding points in the signals can be identified and analyzed.

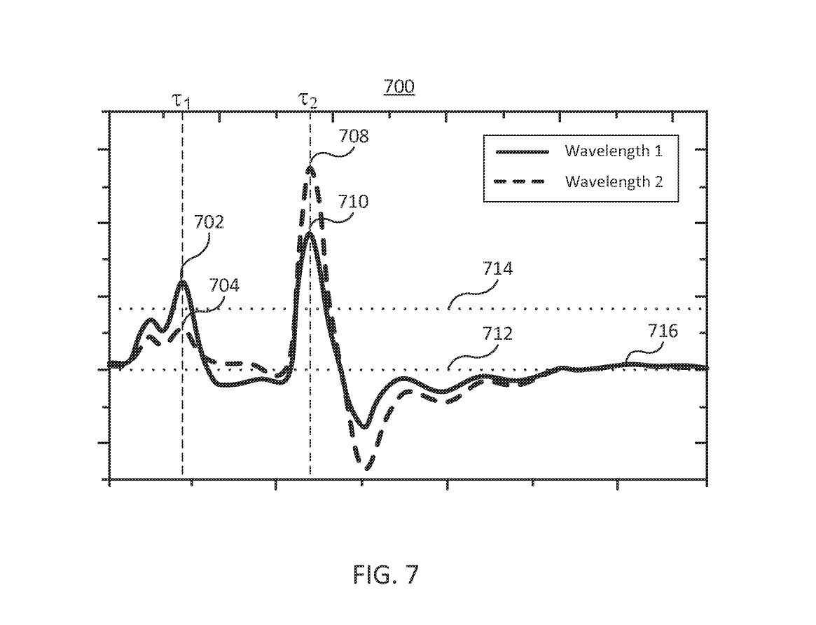

Exemplary aligned photoacoustic signals are shown in FIG. 7. FIG. 7 is an illustrative plot of two photoacoustic signals in accordance with some embodiments of the present disclosure. The photoacoustic signals in plot 700 may be detected, for example, in step 604 (FIG. 6). The system may detect the two photoacoustic signals generated by photonic signals at wavelength 1 and wavelength 2. For example, for a photoacoustic measurement of blood oxygenation, wavelength 1 may be 700 nm light and wavelength 2 may be 800 nm light. The abscissa of plot 700 is presented in units of time relative to the photonic or photoacoustic signal. The abscissa may also be presented in units of distance relative to the path length of the photonic or photoacoustic signal. The ordinate of plot 700 is presented in units of signal intensity. For example, the abscissa may be in microseconds measured from a photonic signal pulse and the ordinate in volts detected by an ultrasonic detector.

The photoacoustic signals shown in plot 700 include multiple peaks, including peak 702 at time τ₁ in response to light of wavelength 1, peak 704 at time τ₁ in response to light of wavelength 2, peak 706 at time τ₂ in response to light of wavelength 1, peak 708 at time τ₂ in response to light of wavelength 2. The intensity of the peaks may be dependent upon the path length, volume of tissue sampled, the characteristics of the sampled tissue, other parameters, and any combination thereof. For example, if peak 702 and peak 704 correspond to the skin of a subject and peak 708 and peak 710 correspond to a large vein of a subject, the large volume of the vein may account for the relatively larger intensity of peak 708 and peak 710. In some embodiments, a peak may correspond to a depth within the subject tissue, and therefore a different part of the subject. For example, a peak received early in time may correspond to a shallow depth, and thus be indicative of the skin, and a peak received later in time may correspond to a deeper depth, and thus correspond to an artery, vein, or other structure.

Referring back to the flow diagram of FIG. 6, in step 606, a first and second peak may be identified based on the acoustic pressure signal. The system may identify the first and second peaks that are the largest two peaks in a photoacoustic signal, that fall within a particular region of the signal, by any other suitable method, or any combination thereof. It will be understood that in some embodiments, the system may identify any number of peaks. In some embodiments, the system may identify peaks using their width, height, shape, other suitable parameters, or any combination thereof.

The system may identify peaks using, in part, a threshold operation. The system may use a threshold such that signal portions below a certain value or values are not considered. For example, referring to FIG. 7, threshold 714 of plot 700 may be used in part to identify peaks. The use of threshold 714 may include peak 708 and exclude peak 716 from subsequent processing. In some embodiments, the threshold may be predetermined by user input or other suitable method. User input may include parameters based in part of the location of the sensor with respect to a subject, the age, height, weight, health, type of sensor in use, wavelengths of light employed for generating a photoacoustic signal, other suitable parameters, or any combination thereof. In some embodiments, the threshold may dynamically adjust based on the noise level, signal strength, number of peaks, spacing of peaks, tissue depth corresponding to the peak, time delay in receiving a peak with respect to photonic signal emission, other suitable ways of thresholding, or any combination thereof. It will be understood that the dynamic threshold may adjust to a single level for a photoacoustic signal over its full range, the dynamic threshold may adjust continuously throughout the photoacoustic signal range, may adjust in sections throughout the photoacoustic signal range, by other suitable schemes, or by any combination thereof.

When the photoacoustic signal includes multiple components corresponding to different photonic signals, peaks may be identified in one component or in multiple components. When peaks are identified in one component, corresponding peaks, points, data, or a combination thereof, may be identified (e.g., based on an alignment process) in the other component or components.

In step 608, the system may determine values indicative of physiological parameters based on the peaks identified in step 606. Peaks in a photoacoustic signal may correspond to a component of a subject that has a relatively higher absorption of the photonic signals (e.g., hemoglobin) and may include information from which physiological parameters can be determined. For example, a peak may correspond to a blood vessel and characteristics of the peak (e.g., amplitude, slope, shape, etc.) may provide information from which physiological parameters may be determined. For example, characteristics of corresponding peaks in a photoacoustic signal generated using photonic signals of two wavelengths may be indicative of oxygen saturation and the concentration of hemoglobin.

In some embodiments, values indicative of physiological parameters may be determined using a time-domain analysis of peaks, for example, the peaks identified in step 606. In some embodiments, the system may in part determine a value corresponding to a peak by integrating the area under the peak (e.g., the peak of an envelope derived from an acoustic detector signal), by determining the height of a peak with respect to the baseline, by determining the position of the peak within the signal, by deconvolving the peak from other peaks, by other suitable processing steps, or by any combination thereof. In some embodiments, a baseline (e.g., baseline 712 in plot 700), may be used to integrate the area below a peak (e.g., peak 708 in plot 700) to determine a physiological value. The baseline may be predetermined by user input, or determined based on the photoacoustic signal (e.g., set at 0, a horizontal line at the average signal level, a horizontal line substantially aligned with the signal level at large time delays, an interpolation of inter-peak signal levels). User input may include parameters based in part of the location of the sensor with respect to a subject, the age, height, weight, health, type of sensor in use, wavelengths of light employed for generating a photoacoustic signal, other suitable parameters, or any combination thereof. Baselines may be determined statically during calibration or setup, dynamically during measurements, by any other suitable technique, or any combination thereof. In some embodiments, the peak-to-peak height may be used to determine values indicative of physiological parameters. The peak-to-peak height refers to the height between a positive and corresponding negative peak in a photoacoustic signal generated from a photoacoustic sensor.

In some embodiments, the values indicative of physiological parameters may be determined in part using peaks from photoacoustic signals corresponding to multiple wavelengths of light. For example, the values indicative of physiological parameters may be determined based on a relationship (e.g., a ratio) between peaks corresponding to different wavelengths of light (e.g., Red and IR). For example, values indicative of oxygen saturation corresponding to the peaks identified in plot 700 may be determined using the techniques described herein. In plot 700, the peaks at τ₁ are more intense for light of wavelength 1 than of wavelength 2, and the peaks at t₂ are more intense for light of wavelength 2 than of wavelength 1. In some embodiments, this may indicate that a physiological parameter is greater for the tissue at a first depth than a second depth.

In some embodiments, any suitable technique or techniques may be used to determine values indicative of physiological parameters from the identified peaks. For example, the techniques disclosed in commonly-assigned Li et al. U.S. patent application Ser. No. 13/284,580, filed Oct. 28, 2011, which is incorporated herein by reference in its entirety, may be used to determine values indicative of physiological parameters from the identified peaks.

Referring back to Eq. 1, the fluence φ(z) may be estimated using techniques such as modeling techniques, oblique-incidence diffuse reflectance (OIR), photon density wave (PDW), other suitable techniques, or any combination thereof. The Grüneisen parameter may be known or assumed. By rearranging Eq. 1, the following equation can be obtained:

$${μ}_{a} = \frac{p ( z )}{Γφ ( z )} \qquad ( 14 )$$

for the absorption coefficient μₐ of the absorbing tissue (hemoglobin of the subject's blood in this example). In some embodiments, the wavelength of the light source may be selected to aid in determining one or more physiological parameters. For example, at a first wavelength λ₁ where oxyhemoglobin and deoxyhemoglobin have approximately the same absorptivity (e.g., around 808 nm), the absorption coefficient α_{a,λ}_{₁} may be given by the following:

μ_{a,λ}_{₁}=tHb·ε_{λ}_{₁}, (15)

where ε_{λ}_{₁} (presumed known) is the absorptivity of the oxyhemoglobin and deoxyhemoglobin at first wavelength λ. Eq. 15 may be solved for tHb from the known μ_{a,λ}_{₁} (e.g., known from using Eq. 14). In some embodiments, a second light source of a second wavelength λ₂, different from the first, may be used to determine blood oxygen saturation. For example, with tHb known, a second absorption coefficient may be determined at the second wavelength. The absorption coefficient μₐ may be given by the following:

μ_{a,λ}_{₂}=ε_{ox,λ}_{₂}cₒₓ+ε_{deox,λ}_{₂}c_{deox}, (16)

where ε_{ox,λ}_{₂} is the absorptivity of oxyhemoglobin, ε_{deox,λ}_{₂} is the absorptivity of deoxyhemoglobin, cₒₓ is the concentration of oxyhemoglobin, and c_{deox} is the concentration of deoxyhemoglobin. The concentration can be related by:

tHb=cₒₓ+c_{deox}, (17)

which may be combined with Eq. 16 to give:

μ_{a,λ}_{₂}=ε_{ox,λ}_{₂}cₒₓ+ε_{deox,λ}_{₂}(tHb−cₒₓ), or (18)

μ_{a,λ}_{₂}=ε_{ox,λ}_{₂}cₒₓ+ε_{deox,λ}_{₂}c_{deox}, (19)

Because tHb is known, any of Eqs. 18 and 19 may be inverted to determine the respective hemoglobin concentration from the known tHb and μ_{a,λ}_{₂}. Additionally, blood oxygen saturation S_{O2} may be determined by the following:

$${S}_{O 2} = \frac{{c}_{ox}}{{c}_{ox} + {c}_{deox}} , \qquad ( 20 )$$

which may be an arterial blood oxygen saturation or venous oxygen saturation depending upon the type of blood vessel. It will be understood that Eqs. 14-20 provide illustrative examples of formulas used to determine values indicative of physiological parameters from photoacoustic measurements. Any suitable equations, models, other suitable mathematical construct, look-up table, database, or other reference may be used to determine one or more physiological parameters based on peaks. For example, in some embodiments, physiological parameters may be tabulated (e.g., in a look-up table stored in encoder 52 of FIG. 2) for discrete values of absorption coefficient at one or more wavelengths. In some embodiments, a pulse rate may be determined based on modulations of detected signals, or parameters derived thereof, at the frequency of the pulse rate. For example, an artery may be monitored, and the pumping of the subject's heart may cause a modulation of detected signals at the frequency of the heart rate.

In step 610, the system may determine one or more physiological parameters based on the values determined in step 608 In some embodiments, the system may compare the determined values indicative of physiological parameters to determine one or more physiological parameters. For example, the values indicative of physiological parameters determined from the peaks of plot 700 in step 608 may indicate that the oxygen saturation may be greater for the peaks at τ₁ and lesser for the peaks at τ₂. In some embodiments, this may indicate that the peaks at τ₂ corresponds to venous blood and that the value determined from the peaks at τ₂ corresponds to venous blood oxygenation. This may also indicate, for example, that the peak at τ₁ corresponds to the skin, given its shallower depth, higher oxygen saturation, and lower intensity. The system may determine, based on an analysis of the values, that the value determined from the peaks at τ₂ corresponds to the venous blood oxygen saturation. The system may use this value as the venous blood oxygen saturation or may convert it (e.g., using a lookup table or one or more equations) to determine the venous blood oxygen saturation.

In some embodiments, the system may determine physiological values using, in part, information (e.g., a total hemoglobin measurement) from an outside source. The information may be received from a third measurement, user input, another measurement device, any other source, or any combination thereof. For example, the system may calculate blood oxygen saturation based on peak heights from two wavelength photonic signals as measured by system 10 of FIG. 1 and the total hemoglobin as measured by a remote device.

In step 610, the system may determine physiological parameters related to different regions of subject tissue. The system may determine that the peak relating to the highest blood oxygen saturation corresponds to the arterial blood oxygen saturation. The system may determine that the peak relating to the lowest blood oxygen saturation corresponds to the venous blood oxygen saturation. The system may therefore determine venous oxygen saturation, arterial oxygen saturation, or both in this way without prior knowledge of which peak corresponds to which blood vessel. It will be understand the step 610 may also be used to determine the concentration of oxyhemoglobin, deoxyhemoglobin, total hemoglobin, or a combination thereof for blood vessels (e.g., arterial and venous blood vessels)

The steps of flow diagram 600 may be implemented, for example, using a sensor that emits two wavelengths of light and that is applied to a location on the neck of a subject. In such an arrangement, the system may detect a photoacoustic signal containing components from the external jugular vein, the external carotid artery, skin, the retromandibular muscle, and other acoustic pressure signal generating structures in response to a photonic signal, as described in step 604. The largest peaks may be identified using a threshold operation, as described in step 606. The area under the peaks may be integrated or otherwise processed to determine values indicative of blood oxygen saturation corresponding to each peak, as described in step 608. The highest oxygen saturation value may correspond to the carotid artery and the lowest oxygen saturation value may correspond to the external jugular vein, as described in step 610.

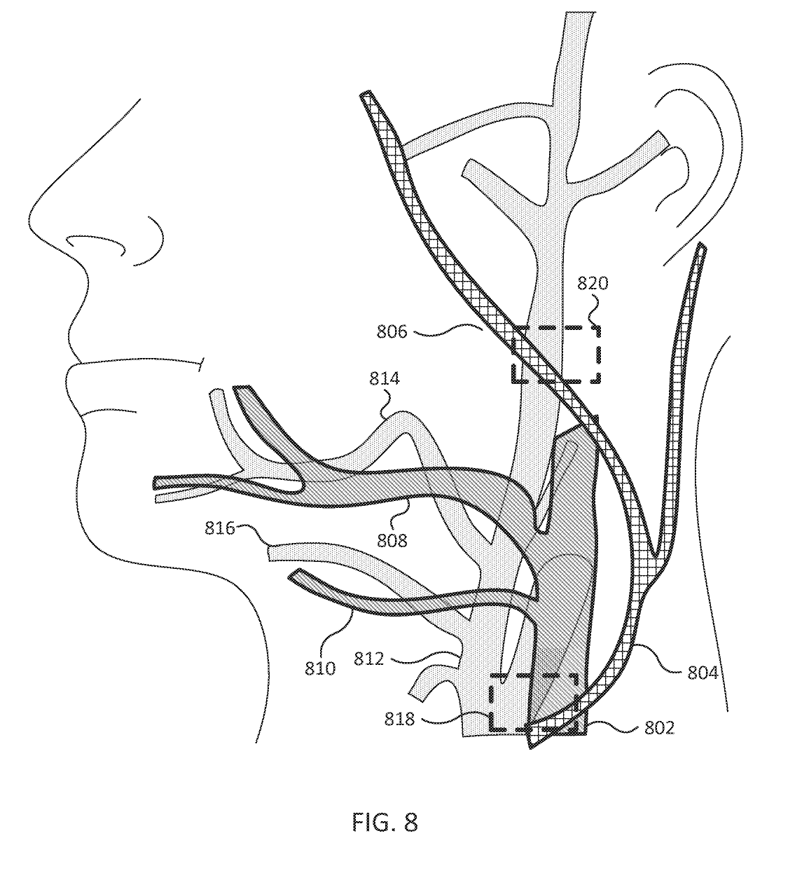

FIG. 8 is an illustrative perspective view of a portion of the circulatory system in the neck of a subject in accordance with some embodiments of the present disclosure. The blood vessels of the neck of a subject may include, in part, internal jugular vein 802, external jugular vein 804, retromandibular vein 806, facial vein 808, lingual vein 810, external carotid artery 812, facial artery 814, lingual artery 816, and other blood vessels and structures.

It will be understood from FIG. 8 that several blood vessels may be proximal to each other in the neck of a typical subject. In some embodiments, a photoacoustic system may detect photoacoustic signals containing components from multiple blood vessels. Those blood vessels typically vary both in size and in expected concentrations of oxyhemoglobin and deoxyhemoglobin. Arterial blood may contain a relatively higher concentration of oxyhemoglobin, while venous blood may contain a relatively lower concentration of oxyhemoglobin. Similarly, arterial blood may contain a relatively lower concentration of deoxyhemoglobin, while venous blood may contain a relatively higher concentration of deoxyhemoglobin. Stated another way, the oxygen saturation of arterial blood is expected to be higher than the oxygen saturation of venous blood. A measurement carried out near the area indicated by region 818, for example, may detect a photoacoustic signal containing components corresponding to the skin, the external carotid artery, external jugular vein, and internal jugular vein. A measurement carried out at the area indicated by region 820 may include a photoacoustic signal related to the skin, the retromandibular vein, and the external carotid artery. Based on the disclosed techniques, the oxygen saturation, the concentration of hemoglobin (e.g., oxygenated, deoxygenated, and/or total hemoglobin), or a combination thereof may be determined for desired arterial and/or venous blood vessels.

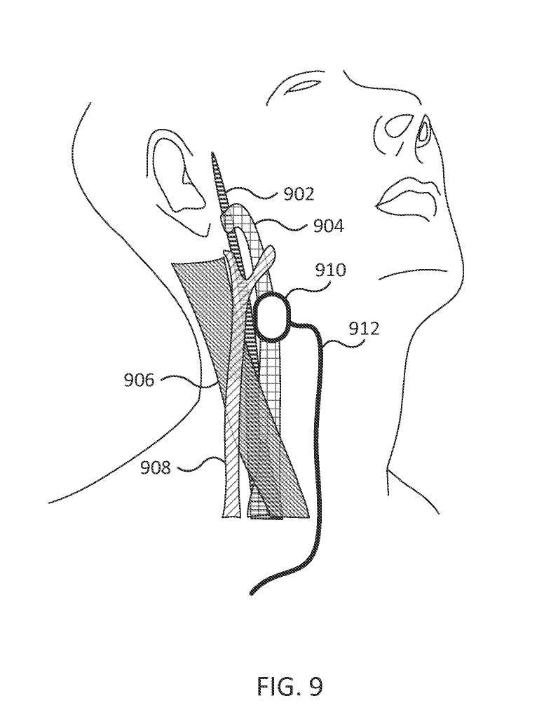

FIG. 9 is another illustrative perspective view of a portion of the circulatory system in the neck of a subject in accordance with some embodiments of the present disclosure. The circulatory system of a typical subject's neck may include external carotid artery 902, internal jugular vein 904, sternocleidomastoid muscle 906, and external jugular vein 908. In some embodiments, photoacoustic detector 910 may be located near to a target area, and may be coupled to remote monitors and processing equipment by connection 912. Connection 912 may be a wired or wireless connection. Connection 912 may also be omitted where some or all of the processing is located within photoacoustic detector 910.

In some embodiments, as illustrated in FIG. 9, photoacoustic detector 910 may be located such that a photoacoustic signal may include primarily the internal jugular vein 904. In the illustrated embodiment, it may be understood that photoacoustic signals may also be generated by external carotid artery 902, sternocleidomastoid muscle 906, and external jugular vein 908. It will be understood that the system may receive signals from different combinations of internal blood vessels and other structures based on the position of detector 910.

It will be understood that the precise size, location, and arrangement of the blood vessels and other structures of the subject may vary between individual subjects. For example, the diameter of the external vein may be larger in an adult than in a child. For example, the position of the external carotid artery in a first subject may be at a different depth with respect to the skin than the depth in a second subject. Therefore, correlating the peaks of a photoacoustic signal with circulatory and other structures based on spatial information alone may be difficult. Accordingly, the disclosed techniques, which use physiological information, enables peaks to be accurately correlated to desired physiological structures in a subject. For example, the peak of a photoacoustic signal corresponding to the external jugular vein may be identified by its saturation value.

In some embodiments, the system may limit the possible spatial positions for a known structure to be identified. For example, if a photoacoustic probe is placed externally on the neck of a subject, the system may expect that the external jugular vein will be located between 1 and 8 mm in depth with respect to the probe surface. In some embodiments, the system may receive user input relating to the general or specific location of the sensor. In some embodiments, the system may receive the location information from the sensor (e.g., based on sensor type).

While the foregoing examples refer to using a photoacoustic sensor on the neck of a subject, it will be understood that the photoacoustic sensor may applied to any suitable location on a subject.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A photoacoustic system for determining a physiological parameter, the system comprising:
a light source configured to provide a photonic signal to a subject;
an acoustic detector configured to detect an acoustic pressure signal from the subject, wherein the acoustic pressure signal is caused by absorption of at least some of the photonic signal by the subject; and
processing equipment communicatively coupled to the acoustic detector, the processing equipment configured to:
identify first and second peaks based on the acoustic pressure signal;
determine first and second values indicative of a physiological parameter, wherein the first value corresponds to the first peak and the second value corresponds to the second peak; and
determine the physiological parameter based on the first and second values.

2. The system of claim 1, wherein the first peak corresponds to an arterial blood vessel and the second peak corresponds to a venous blood vessel.

3. The system of claim 2, wherein the first and second values indicative of a physiological parameter are indicative of oxygen saturation.

4. The system of claim 3, wherein the processing equipment is further configured to:
analyze the first and second values; and
select a value corresponding to a lower oxygen saturation, wherein the determined physiological parameter is venous oxygen saturation.

5. The system of claim 3, wherein the processing equipment is further configured to:
analyze the first and second values; and
select a value corresponding to a higher oxygen saturation, wherein the determined physiological parameter is arterial oxygen saturation.

6. The system of claim 1, wherein the first and second values indicative of a physiological parameter are indicative of hemoglobin concentration.

7. The system of claim 1, wherein the processing equipment is further configured to identify the first and second peaks based on an analysis of the acoustic pressure signal and at least one threshold.

8. The system of claim 1, wherein the light source comprises one or more emitters and wherein the photonic signal comprises light of two different wavelengths between 600 nm and 1000 nm.

9. The system of claim 8, wherein the acoustic pressure signal comprises a first component corresponding to a first of the two different wavelengths and a second component corresponding to a second of the two different wavelengths, and wherein the processing equipment is further configured to determine the first and second values based on the first and second components.

10. The system of claim 8, wherein the one or more emitters emit the light of the two different wavelengths at different times.

11. A photoacoustic method for determining a physiological parameter, the method comprising:
providing a photonic signal to a subject from a light source;
detecting an acoustic pressure signal using an acoustic detector, wherein the acoustic pressure signal is caused by absorption of at least some of the photonic signal by the subject;
identifying first and second peaks based on the acoustic pressure signal;
determining first and second values indicative of a physiological parameter, wherein the first value corresponds to the first peak and the second value corresponds to the second peak; and
determining the physiological parameter based on the first and second values.

12. The method of claim 11, wherein identifying the first peak comprises the first peak corresponding to an arterial blood vessel and identifying the second peak comprises the second peak corresponding to a venous blood vessel.

13. The method of claim 12, wherein determining the first and second values indicative of a physiological parameter further comprises determining first and second values indicative of oxygen saturation.

14. The method of claim 13, further comprising:
analyzing the first and second values; and
selecting a value corresponding to a lower oxygen saturation, wherein the determined physiological parameter is venous oxygen saturation.

15. The method of claim 13, further comprising:
analyzing the first and second values; and
selecting a value corresponding to a higher oxygen saturation, wherein the determined physiological parameter is arterial oxygen saturation.

16. The method of claim 11, wherein determining the first and second values indicative of a physiological parameter further comprises determining first and second values indicative of hemoglobin concentration.

17. The method of claim 11, further comprising identifying the first and second peaks based on an analysis of the acoustic pressure signal and at least one threshold.

18. The method of claim 11, wherein the light source comprises one or more emitters and wherein the photonic signal comprises light of two different wavelengths between 600 nm and 1000 nm.

19. The method of claim 18, wherein detecting the acoustic pressure signal further comprises detecting a first component corresponding to a first of the two different wavelengths and a second component corresponding to a second of the two different wavelengths, and wherein the first and second values are based on the first and second components.

20. The method of claim 18, wherein the one or more emitters emit light of the two different wavelengths at different times.
